(19) United States
(12) Patent Application Publication
(10) Pub. No.: US 2024/0151734 A1
(43) Pub. Date: May 9, 2024

(54) SYSTEMS AND METHODS FOR IDENTIFYING BLOOD CONDITIONS VIA DOT PLOT ANALYSIS

(71) Applicant: IDEXX Laboratories, Inc., Westbrook, ME (US)

(72) Inventors: Jeremy Hammond, Standish, ME (US); Heidi Peta, Edmonton (CA); James Russell, North Yarmouth, ME (US); Dennis DeNicola, Windham, ME (US)

(21) Appl. No.: 18/387,186

(22) Filed: Nov. 6, 2023

Related U.S. Application Data
(60) Provisional application No. 63/423,204, filed on Nov. 7, 2022.

(51) Int. Cl. G01N 33/80; B01L 3/00

(52) U.S. Cl. CPC G01N 33/80; B01L 3/502715; B01L 2200/16; B01L 2300/0654; B01L 2300/0877

(57) ABSTRACT

Disclosed are approaches for analyzing a two-dimensional (2D) dot plot, without human intervention, to identify conditions in a hematology sample. The analyses operate to provide indications of left shift and/or small-pathologic red blood cells based on spatial distribution of one or more groups of dots in the 2D dot plot. Spatial distribution of a group of white blood cell dots is analyzed to provide an indication of left shift. Spatial distribution of a group of red blood cell dots is analyzed to provide an indication of presence of small-pathologic red blood cells.

### CROSS-REFERENCE TO RELATED APPLICATIONS

This application claims the benefit of, and priority to, U.S. Provisional Patent Application No. 63/423,204, filed on Nov. 7, 2022, the entire contents of which are hereby incorporated herein by reference.

### TECHNICAL FIELD

The present disclosure relates to hematology analyzers, and more particularly, to analyzing dot plots generated by hematology analyzers to assist with identifying various blood conditions.

### BACKGROUND

Hematology analyzers can be utilized to count and identify blood cells. For example, hematology analyzers can detect and count different types of blood cells and can identify anomalies within blood samples.

### SUMMARY

As one example, flow cytometers are hematology analyzers that measure components such as cells and particles in a solution, which move along a cuvette in front of a light source (e.g., a laser) in a single file. Light from the light source is absorbed and scattered by the components in a manner that is dictated by associated stains in the solution and/or the size and morphology of the components.

As another example, some hematology analyzers utilize image recognition to interrogate cells in a blood sample. For example, blood cells can be arranged in a single layer on a cartridge or the like, and images can be taken of the blood cells. The images, in embodiments, are analyzed to determine the size and morphology of the components, and/or to determine the fluorescent response of the components.

The present disclosure provides systems, methods, and instructions for analyzing a two-dimensional (2D) dot plot, without human intervention, to identify conditions in a blood sample. In particular, embodiments of the analyses of the present disclosure operate to provide indications of left shift or small-pathologic red blood cells.

In accordance with aspects of the present disclosure, a system for identifying conditions in hematology samples includes at least one processor and at least one memory storing instructions. The instructions, when executed by the at least one processor, cause the system to, without human intervention: process and identify constituents in a hematology sample; determine a two-dimensional (2D) dot plot corresponding to the identified constituents in the hematology sample, where the 2D dot plot has a complexity axis indicative of complexity of the constituents in the hematology sample and a size axis indicative of size of the constituents in the hematology sample; analyze the 2D dot plot to determine a spatial distribution of a group of dots in the 2D dot plot corresponding to at least a portion of white blood cells in the hematology sample; and based on the spatial distribution of the group of dots in the 2D dot plot, provide an indication of left shift.

In embodiments of the system, in analyzing the 2D dot plot, the instructions, when executed by the at least one processor, cause the system to, without human intervention, perform at least one analysis among a plurality of analyses that include: determining orientation and dimensions of a geometric shape that surrounds the group of dots in the 2D dot plot, determining a centroid of the group of dots relative to a configurable location in the 2D dot plot where a centroid of dots corresponding to healthy populations of corresponding white blood cells would be located in the 2D dot plot, determining mean and standard deviation of the group of dots with respect to one of: the complexity axis or the size axis, and determining spatial density bands of the group of dots in the 2D dot plot.

In embodiments of the system, in analyzing the 2D dot plot, the instructions, when executed by the at least one processor, cause the system to, without human intervention, perform each analysis in the plurality of analyses.

In embodiments of the system, in providing the indication, the instructions, when executed by the at least one processor, cause the system to, without human intervention: compare an angle, formed by a major axis of the geometric shape and the complexity axis of the 2D dot plot, to a configurable angle threshold; and determine presence of left shift based on the angle being greater than the configurable angle threshold.

In embodiments of the system, the configurable location in the 2D dot plot has a complexity-axis value and a size-axis value. In providing the indication, the instructions, when executed by the at least one processor, cause the system to, without human intervention: determine direction and magnitude of a vector from the configurable location to the centroid of the group of dots; and determine presence of left shift based on: (1) the direction of the vector being directed towards one or both of: a lesser-value side of the complexity-axis value and a greater-value side of the size-axis value, and (2) the magnitude of the vector being greater than a configurable magnitude threshold.

In embodiments of the system, in providing the indication, the instructions, when executed by the at least one processor, cause the system to, without human intervention, determine presence of left shift based on at least one of: a complexity-axis standard deviation of the group of dots being less than a configurable complexity-axis standard deviation threshold, or a size-axis standard deviation of the group of dots being greater than a configurable size-axis standard deviation threshold.

In embodiments of the system, in providing the indication, the instructions, when executed by the at least one processor, cause the system to, without human intervention, determine presence of left shift based on a designated low-density band among the spatial density bands defining at least one of: a height evaluated along the size axis greater than a configurable size height threshold or a width evaluated along the complexity axis less than a configurable complexity width threshold.

In accordance with aspects of the present disclosure, a processor-implemented method for identifying conditions in hematology samples includes, without human intervention: processing and identifying constituents in a hematology sample; determining a two-dimensional (2D) dot plot corresponding to the identified constituents of the hematology sample, where the 2D dot plot has a complexity axis indicative of complexity of the constituents in the hematology sample and a size axis indicative of size of the constituents in the hematology sample; analyzing the 2D dot plot to determine a spatial distribution of a group of dots in the 2D dot plot corresponding to at least a portion of white blood cells in the hematology sample; and based on the spatial distribution of the group of dots in the 2D dot plot, providing an indication of left shift.

In embodiments of the processor-implemented method, analyzing the 2D dot plot includes, without human intervention, performing at least one analysis among a plurality of analyses that include: determining orientation and dimensions of a geometric shape that surrounds the group of dots in the 2D dot plot, determining a centroid of the group of dots relative to a configurable location in the 2D dot plot where a centroid of dots corresponding to healthy populations of corresponding white blood cells would be located in the 2D dot plot, determining mean and standard deviation of the group of dots with respect to one of: the complexity axis or the size axis, and determining spatial density bands of the group of dots in the 2D dot plot.

In embodiments of the processor-implemented method, analyzing the 2D dot plot includes, without human intervention, performing each analysis in the plurality of analyses.

In embodiments of the processor-implemented method, providing the indication includes, without human intervention: comparing an angle, formed by a major axis of the geometric shape and the complexity axis of the 2D dot plot, to a configurable angle threshold; and determining presence of left shift based on the angle being greater than the configurable angle threshold.

In embodiments of the processor-implemented method, the configurable location in the 2D dot plot has a complexity-axis value and a size-axis value. Providing the indication includes, without human intervention: determining direction and magnitude of a vector from the configurable location to the centroid of the group of dots; and determining presence of left shift based on: (1) the direction of the vector being directed towards one or both of: a lesser-value side of the complexity-axis value and a greater-value side of the size-axis value, and (2) the magnitude of the vector being greater than a configurable magnitude threshold.

In embodiments of the processor-implemented method, providing the indication includes, without human intervention: determining presence of left shift based on at least one of, a complexity-axis standard deviation of the group of dots being less than a configurable complexity-axis standard deviation threshold, or a size-axis standard deviation of the group of dots being greater than a configurable size-axis standard deviation threshold.

In embodiments of the processor-implemented method, providing the indication includes, without human intervention, determining presence of left shift based on a designated low-density band among the spatial density bands defining at least one of: a height evaluated along the size axis greater than a configurable size height threshold or a width evaluated along the complexity axis less than a configurable complexity width threshold.

In accordance with aspects of the present disclosure, a non-transitory process-readable medium stores instructions which, when executed by at least one processor of a system, cause the system to, without human intervention: process and identify constituents in a hematology sample; determine a two-dimensional (2D) dot plot corresponding to the identified constituents in the hematology sample, where the 2D dot plot has a complexity axis indicative of complexity of the constituents in the hematology sample and a size axis indicative of size of the constituents in the hematology sample; analyze the 2D dot plot to determine a spatial distribution of a group of dots in the 2D dot plot corresponding to at least a portion of white blood cells in the hematology sample; and based on the spatial distribution of the group of dots in the 2D dot plot, provide an indication of left shift.

In embodiments of the non-transitory processor-readable medium, in analyzing the 2D dot plot, the instructions, when executed by the at least one processor, cause the system to, without human intervention, perform at least one analysis among a plurality of analyses that include: determining orientation and dimensions of a geometric shape that surrounds the group of dots in the 2D dot plot, determining a centroid of the group of dots relative to a configurable location in the 2D dot plot where a centroid of dots corresponding to healthy populations of corresponding white blood cells would be located in the 2D dot plot, determining mean and standard deviation of the group of dots with respect to one of: the complexity axis or the size axis, and determining spatial density bands of the group of dots in the 2D dot plot.

In embodiments of the non-transitory processor-readable medium, in analyzing the 2D dot plot, the instructions, when executed by the at least one processor, cause the system to, without human intervention, perform each analysis in the plurality of analyses.

In embodiments of the non-transitory processor-readable medium, in providing the indication, the instructions, when executed by the at least one processor, cause the system to, without human intervention: compare an angle, formed by a major axis of the geometric shape and the complexity axis of the 2D dot plot, to a configurable angle threshold; and determine presence of left shift based on the angle being greater than the configurable angle threshold.

In embodiments of the non-transitory processor-readable medium, the configurable location in the 2D dot plot has a complexity-axis value and a size-axis value. In providing the indication, the instructions, when executed by the at least one processor, cause the system to, without human intervention: determine direction and magnitude of a vector from the configurable location to the centroid of the group of dots; and determine presence of left shift based on: (1) the direction of the vector being directed towards one or both of: a lesser-value side of the complexity-axis value and a greater-value side of the size-axis value, and (2) the magnitude of the vector being greater than a configurable magnitude threshold.

In embodiments of the non-transitory processor-readable medium, in providing the indication, the instructions, when executed by the at least one processor, cause the system to, without human intervention, determine presence of left shift based on at least one of: a complexity-axis standard deviation of the group of dots being less than a configurable complexity-axis standard deviation threshold, or a size-axis standard deviation of the group of dots being greater than a configurable size-axis standard deviation threshold.

In embodiments of the non-transitory processor-readable medium, in providing the indication, the instructions, when executed by the at least one processor, cause the system to, without human intervention, determine presence of left shift based on a designated low-density band among the spatial density bands defining at least one of: a height evaluated along the size axis greater than a configurable size height threshold or a width evaluated along the complexity axis less than a configurable complexity width threshold.

In accordance with aspects of the present disclosure, a system for identifying conditions in hematology samples includes: at least one processor and at least one memory storing instructions. The instructions, when executed by the at least one processor, cause the system to, without human intervention: process and identify constituents in a hematology sample; determine a two-dimensional (2D) dot plot corresponding to the identified constituents in the hematology sample, where the 2D dot plot has a separate axis indicative of at least one of complexity or fluorescence of the constituents in the hematology sample and a size axis indicative of size of the constituents in the hematology sample; analyze the 2D dot plot to determine a spatial distribution of a group of dots in the 2D dot plot corresponding to at least a portion of red blood cells in the hematology sample; and based on the spatial distribution of the group of dots in the 2D dot plot, provide an indication of presence of small pathologic red blood cells.

In embodiments of the system, in analyzing the 2D dot plot, the instructions, when executed by the at least one processor, cause the system to, without human intervention, perform at least one analysis among a plurality of analyses that includes: determining a centroid of the group of dots relative to a configurable location in the 2D dot plot where a centroid of dots corresponding to healthy populations of corresponding red blood cells would be located in the 2D dot plot, determining a size-axis distribution of a subset of the group of dots below a mean of the group of dots evaluated along the size axis, and determining spatial density bands of the group of dots in the 2D dot plot.

In embodiments of the system, in analyzing the 2D dot plot, the instructions, when executed by the at least one processor, cause the system to, without human intervention, perform each analysis in the plurality of analyses.

In embodiments of the system, the configurable location in the 2D dot plot has a separate-axis value and a size-axis value. In providing the indication, the instructions, when executed by the at least one processor, cause the system to, without human intervention, determine presence of small pathologic red blood cells based on: (1) the direction of the vector being directed towards a lesser-value side of the size-axis value; and (2) the magnitude of the vector being greater than a configurable magnitude threshold.

In embodiments of the system, in providing the indication, the instructions, when executed by the at least one processor, cause the system to, without human intervention, determine presence of small pathologic red blood cells based on the size-axis distribution being greater than a configurable size-axis distribution threshold.

In embodiments of the system, in providing the indication, the instructions, when executed by the at least one processor, cause the system to, without human intervention, determine presence of small pathologic red blood cells based on a designated low-density band among the spatial density bands extending below the mean of the group of dots evaluated along the size axis by a height more than a configurable height threshold.

In accordance with aspects of the present disclosure, a processor-implemented method for identifying conditions in hematology samples includes, without human intervention: processing and identifying constituents in a hematology sample; determining a two-dimensional (2D) dot plot corresponding to the identified constituents in the hematology sample, where the 2D dot plot has a separate axis indicative of at least one of complexity or fluorescence of the constituents in the hematology sample and a size axis indicative of size of the constituents in the hematology sample; analyzing the 2D dot plot to determine a spatial distribution of a group of dots in the 2D dot plot corresponding to at least a portion of red blood cells in the hematology sample; and based on the spatial distribution of the group of dots in the 2D dot plot, providing an indication of small pathologic red blood cells.

In embodiments of the processor-implemented method, analyzing the 2D dot plot includes, without human intervention, performing at least one analysis among a plurality of analyses that include: determining a centroid of the group of dots around a configurable location in the 2D dot plot where a centroid of dots corresponding to healthy populations of corresponding red blood cells would be located in the 2D dot plot, determining a size-axis distribution of a subset of the group of dots below a mean of the group of dots evaluated along the size axis, and determining spatial density bands of the group of dots in the 2D dot plot.

In embodiments of the processor-implemented method, analyzing the 2D dot plot includes, without human intervention, performing each analysis in the plurality of analyses.

In embodiments of the processor-implemented method, the configurable location in the 2D dot plot has a separate-axis value and a size-axis value. Providing the indication includes, without human intervention: determining direction and magnitude of a vector from the configurable location to the centroid of the group of dots; and determining presence of small pathologic red blood cells based on: (1) the direction of the vector being towards a lesser-value side of the size-axis value, and (2) the magnitude of the vector being greater than a configurable magnitude threshold.

In embodiments of the processor-implemented method, providing the indication includes, without human intervention, determining presence of small pathologic red blood cells based on the size-axis distribution of the group of dots being greater than a configurable size-axis distribution threshold.

In embodiments of the processor-implemented method, providing the indication includes, without human intervention, determining presence of small pathologic red blood cells based on a designated low-density band among the spatial density bands extending below the mean of the group of dots evaluated along the size axis by a height more than a configurable height threshold.

In accordance with aspects of the present disclosure, a non-transitory processor-readable storage medium stores instructions which, when executed by at least one processor of a system, cause the system to, without human intervention: process and identify constituents in a hematology sample; determine a two-dimensional (2D) dot plot corresponding to the identified constituents in the hematology sample, where the 2D dot plot has a separate axis indicative of at least one of complexity or fluorescence of the constituents in the hematology sample and a size axis indicative of size of the constituents in the hematology sample; analyze the 2D dot plot to determine a spatial distribution of a group of dots in the 2D dot plot corresponding to at least a portion of red blood cells in the hematology sample; and based on the spatial distribution of the group of dots in the 2D dot plot, provide an indication of presence of small pathologic red blood cells.

In embodiments of the non-transitory processor-readable medium, in analyzing the 2D dot plot, the instructions, when executed by the at least one processor, cause the system to, without human intervention, perform at least one analysis among a plurality of analyses that include: determining a centroid of the group of dots around a configurable location in the 2D dot plot where a centroid of dots corresponding to healthy populations of corresponding red blood cells would be located in the 2D dot plot, determining a size-axis distribution of a subset of the group of dots below a mean of the group of dots evaluated along the size axis, and determining spatial density bands of the group of dots in the 2D dot plot.

In embodiments of the non-transitory processor-readable medium, in analyzing the 2D dot plot, the instructions, when executed by the at least one processor, cause the system to, without human intervention, perform each analysis in the plurality of analyses.

In embodiments of the non-transitory processor-readable medium, the configurable location in the 2D dot plot has a separate-axis value and a size-axis value. In providing the indication, the instructions, when executed by the at least one processor, cause the system to, without human intervention: determine direction and magnitude of a vector from the configurable location to the centroid of the group of dots; and determine presence of small pathologic red blood cells based on: (1) the direction of the vector being directed towards a lesser-value side of the size-axis value, and (2) the magnitude of the vector being greater than a configurable magnitude threshold.

In embodiments of the non-transitory processor-readable medium, in providing the indication, the instructions, when executed by the at least one processor, cause the system to, without human intervention, determine presence of small pathologic red blood cells based on the size-axis distribution being greater than a configurable size-axis distribution threshold.

In embodiments of the non-transitory processor-readable medium, in providing the indication, the instructions, when executed by the at least one processor, cause the system to, without human intervention, determine presence of small pathologic red blood cells based on a designated low-density band among the spatial density bands extending below the mean of the group of dots evaluated along the size axis by a height more than a configurable height threshold.

The details of one or more embodiments of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A detailed description of embodiments of the disclosure will be made with reference to the accompanying drawings, wherein like numerals designate corresponding parts in the figures:

FIG. 1 is a diagram of an exemplary hematology system, in accordance with aspects of the present disclosure;

FIGS. 2A and 2B are diagrams of other exemplary hematology systems, in accordance with aspects of the present disclosure;

FIG. 3 is a block diagram of an exemplary hematology system, in accordance with aspects of the present disclosure;

FIG. 4 is a diagram of an exemplary two-dimensional (2D) dot plot relating to white blood cells in a patient without left shift, in accordance with aspects of the present disclosure;

FIG. 5 is a diagram of an exemplary 2D dot plot relating to red blood cells, platelets, and reticulocytes in a patient without small-pathologic red blood cells, in accordance with aspects of the present disclosure;

FIG. 6 is a diagram of an exemplary 2D dot plot relating to white blood cells in a patient with left shift, in accordance with aspects of the present disclosure;

FIGS. 7A and 7B are diagrams of exemplary 2D dot plots relating to analysis of neutrophil dots based on a geometric shape, in accordance with aspects of the present disclosure;

FIG. 8 is a diagram of an exemplary 2D dot plot relating to an analysis of neutrophil dots based on centroids, in accordance with aspects of the present disclosure;

FIG. 9 is a diagram of an exemplary 2D dot plot relating to an analysis of neutrophil dots based on standard deviation, in accordance with aspects of the present disclosure;

FIG. 10 is a diagram of an exemplary 2D dot plot relating to an analysis of neutrophil dots based on density bands, in accordance with aspects of the present disclosure;

FIG. 11 is a diagram of an exemplary 2D dot plot relating to red blood cells, platelets, and reticulocytes in a patient with small-pathologic red blood cells, in accordance with aspects of the present disclosure;

FIG. 12 is a diagram of an exemplary 2D dot plot relating to an analysis of red blood cell dots based on centroids, in accordance with aspects of the present disclosure;

FIG. 13 is a diagram of an exemplary 2D dot plot relating to an analysis of red blood cell dots based on standard deviation, in accordance with aspects of the present disclosure;

FIG. 14 is a diagram of an exemplary 2D dot plot relating to an analysis of red blood cell dots based on density bands, in accordance with aspects of the present disclosure;

FIG. 15 is a flow chart of an exemplary operation for providing an indication of left shift, in accordance with aspects of the present disclosure; and

FIG. 16 is a flow chart of an exemplary operation for providing an indication of presence of small-pathologic red blood cells, in accordance with aspects of the present disclosure.

### DETAILED DESCRIPTION

The present disclosure provides systems, methods, and instructions for analyzing a two-dimensional (2D) dot plot, without human intervention, to assist in identifying left shift or small-pathologic red blood cells in a blood sample.

As used herein, the term “exemplary” does not necessarily mean “preferred” and may simply refer to an example unless the context clearly indicates otherwise. Although the disclosure is not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like.

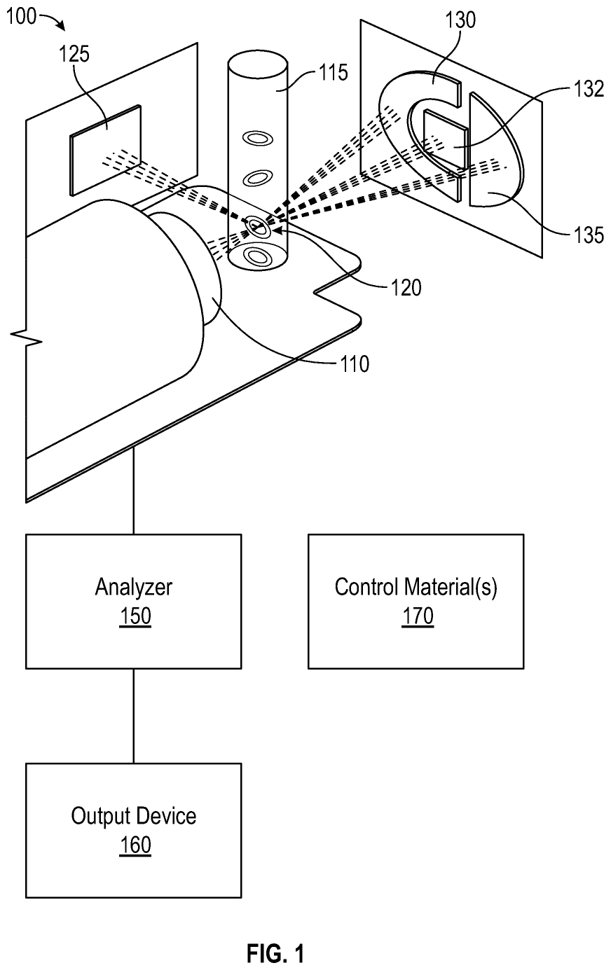

FIG. 1 illustrates a schematic view of a hematology system 100 according to aspects of the present disclosure. As used herein, the terms “hematology system” and “hematology analyzer” may be used interchangeably. The depicted hematology system 100 is configured as a flow cytometry system including a light source 110, an analyzer 150, and an output device 160. A quality control (QC) material 170 can be utilized with the system 100, as described in greater detail herein. The hematology system 100, in embodiments, includes a cuvette/flow cell 115 and light sensors 125, 130, 132, and 135. The components are illustrated for explanatory purposes and are not drawn to scale.

In operation, as a hematology sample's constituents 120 (e.g., cells) move through the cuvette/flow cell 115, the light source 110 emits a beam of light that is oriented transverse to the axial flow of the sample's constituents 120 through the cuvette/flow cell 115. The beam of light emitted by the light source 110 has a central axis. In embodiments, the beam can be a focused narrow band beam (e.g., a laser) or can be a broadband beam. Aspects of exemplary hematology systems are described in U.S. Pat. Nos. 6,320,656, 6,618,143, and 6,784,981, which are hereby incorporated by reference herein in their entireties. A brief description is provided below.

In examples, a portion of the beam from the light source 110 that impinges upon the sample's constituents 120 (e.g., the cells) flowing in the cuvette/flow cell 115 is scattered at a right angle or substantially a right angle to the central axis of the beam of light (side scattered light, denoted as “SS”) and is sensed/measured by the SS sensor 125. As used herein, the term “substantially a right angle” means and includes scattered light which is sensed/measured by SS sensor 125, even though it may not be scattered at exactly a right angle. With respect to light scattered in the hematology systems described herein, any angle with respect to an axis means and includes such angle in any plane that includes the entire axis, without regard to the direction of the angle (e.g., 3° above an axis and 3° below an axis are both encompassed). As persons skilled in the art will understand, an infinite number of planes wholly include an axis, and an angle as used herein may be in any such plane.

Another portion of the beam from the light source 110 that impinges upon the constituents flowing in the cuvette/flow cell 115 is scattered at a much lower angle than 90° with respect to the central axis of the beam of light. This scatter is termed “low angle forward scattered light” (FSL) and has an angle range, for example, between approximately 1° to approximately 3° from the central axis of the beam from the light source 110, inclusive of the endpoints, or can have another angle range that persons skilled in the art will recognize. In the illustrated embodiment, the FSL sensor 135 is oriented to capture/measure the low angle forward scatter light and is oriented at approximately 1° to approximately 3° from the central axis of the beam of the light source 110, inclusive of the endpoints.

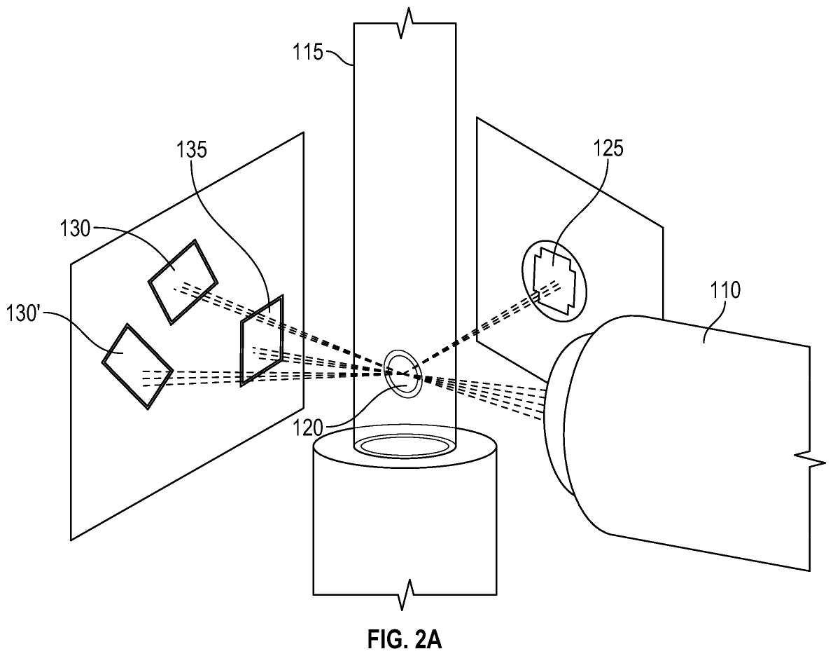

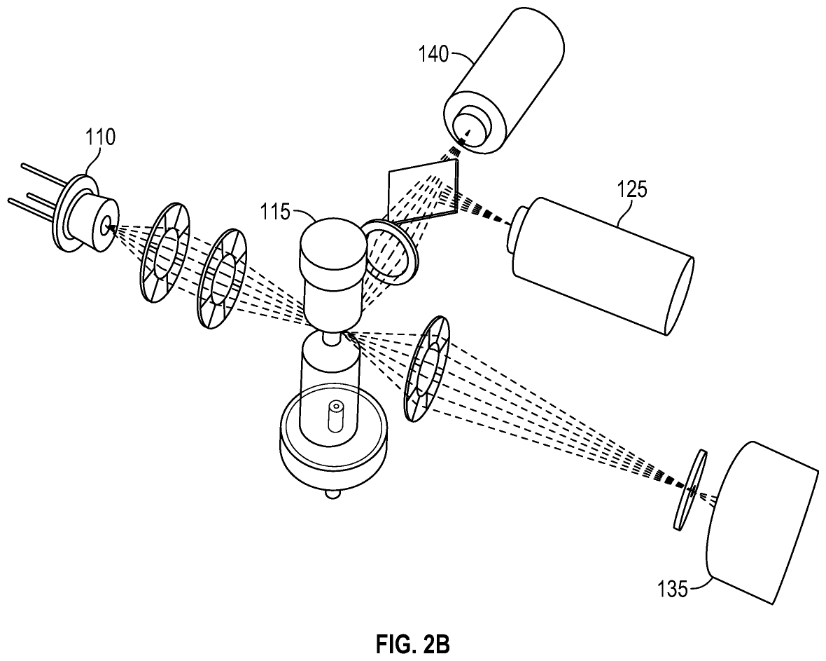

In the hematology system 100, various other light may be sensed/measured, and persons skilled in the art will recognize them. In embodiments, such other light include extinction/axial light (EXT) (e.g., from approximately 0° to approximately 0.5°, inclusive of the endpoints), which is sensed/measured by the E×T sensor 132, and high angle forward scattered light (FSH) (e.g., from approximately 4° to approximately 9°, inclusive of the endpoints), which is sensed/measured by the FSH sensor 130. Such light and angle ranges are exemplary, and other light and other angle ranges will be recognized by persons skilled in the art. In embodiments, a time metric called time-of-flight (TOF) may be measured and analyzed. As persons skilled in the art will recognize, TOF refers to the amount of time that a sample's constituent (e.g., a cell) is interrogated by the beam from the light source 110. TOF may be determined based on EXT light sensed/measured by E×T sensor 132. In embodiments, fluorescence light may be sensed/measured (e.g., FIG. 2B). The disclosure below may refer to one or more of SS, FSL, FSH, EXT, TOF, and fluorescence, as examples of light and metrics that can be used in accordance with aspects of the present disclosure. It is intended and will be understood that other flow cytometry and/or hematology system signals and metrics not expressly mentioned herein are also encompassed within the scope of the present disclosure.

The configuration of sensors 125, 130, 132, 135 in FIG. 1 is exemplary. In embodiments, a flow cytometry hematology system may be implemented by other configurations of sensors or components different from those shown in FIG. 1. An example of another possible sensor configuration is shown in FIG. 2A, which includes the light source 110, the flow cell 115, the SS sensor 125, FSH sensor(s) 130 and 130′, and FSL sensor 135. Light from the light source 110 that interact with a sample's constituents 120 (e.g., cells) are sensed/measured by the sensors 130, 130′, 125, and 135, similar to the configuration shown in FIG. 1. However, in the example depicted in FIG. 2A, no E×T sensor 132 (FIG. 1) is included.

Another example of a possible sensor configuration is shown in FIG. 2B, which includes the light source 110, the flow cell 115, the SS sensor 125, the FSL sensor 135, and a fluorescence sensor 140. Light from the light source 110 interacts with a sample's constituents (e.g., cells) and associated fluorescence is sensed/measured by the sensors 125, 135, and 140. Such and other embodiments are contemplated to be within the scope of the present disclosure. The disclosure below may refer to the sensor configuration of FIG. 1 as an example of a configuration that may be used in accordance with aspects of the present disclosure. It is intended and understood that other sensor configurations, such as the configurations of FIGS. 2A and 2B or any other suitable configuration of light and/or other sensors, may also be used and are also encompassed within the scope of the present disclosure.

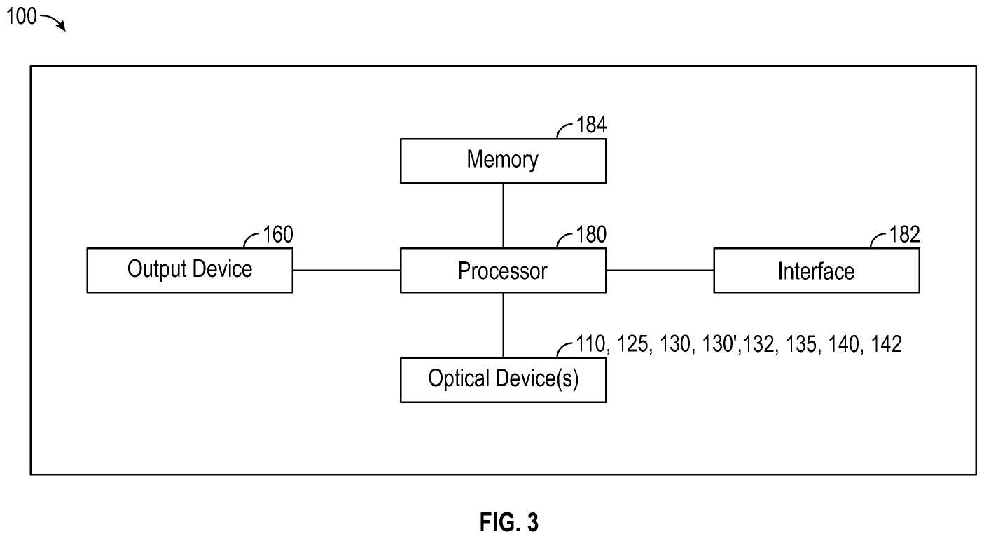

Referring to FIG. 3, a block diagram of the hematology system 100 is depicted. In embodiments, the hematology system 100 includes a processor 180, a memory 184, and the output device 160. In embodiments, the processor 180 and memory 184 may form the analyzer 150 shown in FIG. 1. In embodiments, the processor 180 may be any type of computing device, such as a microprocessor, a microcontroller, or a digital signal processor, among other computing devices, such as others mentioned later herein. The output device 160 can include a graphical user interface (GUI), a screen, one or more devices in communication with the processor 180 (such as smartphones, tablets, phablets), and/or any other device or interface suitable for displaying data. In some embodiments, the hematology system 100 includes a user interface 182 configured to receive user input. The user interface 182 may include a GUI, an alpha-numeric keyboard, one or more remote devices in communication with the processor (such as smartphones, tablets, phablets), and/or any other suitable device, devices interface, or interfaces suitable for receiving user input. While the hematology system 100 is depicted as including a single memory 184 and processor 180, it should be understood that this is merely an example, and the hematology system 100 can include any suitable number of processors 180 and volatile or non-volatile memory 184.

The processor 180, in embodiments, is communicatively coupled to one or more optical devices, such as the light source 110 and the one or more sensors 125, 130, 130′, 135, and 140 as described above and depicted in FIGS. 1, 2A, and 2B. The processor 180 can send and/or receive signals from the light source 110 and the one or more sensors 125, 130, 130′, 135, and 140. In embodiments, the processor 180 is configured to execute instructions to perform various analyses and implement various operations described herein, and such instructions may be stored in the memory 184 and/or in storage media (not shown) of the hematology system 100. Persons skilled in the art will understand how to implement and use the memory 184, storage media, instructions, and processor(s) 180. In embodiments, the hematology system 100 may include one or more application specific integrated circuits (ASIC) and/or other hardware, such as printed circuit boards, among other things.

Referring to FIGS. 1-3, the one or more sensors 125, 130, 130′, 135, and 140 provide signals corresponding to sensed/measured light. Such signals may be referred to generally as “sensor signals” and may be referred to more specifically as, e.g., SS sensor signal, FSL sensor signal, etc. The processor 180 converts the sensor signals to data (e.g., digital values) indicative of, for example, amounts of SS, FSL, FSH, EXT, fluorescence, or other light, sensed/measured by sensors, or indicative of TOF or other metrics. As used herein, data converted from sensor signals will be referred to generally as “sensed data” and may be referred to more specifically as, e.g., sensed SS data, sensed FSL data, etc.

In some embodiments, such as embodiments in which the hematology system 100 utilizes image data, the one or more optical devices include an imager 142 communicatively coupled to the processor 180, as shown in FIG. 3. The imager 142, in embodiments, includes a microscopic camera or the like configured to take images of a blood sample. In some embodiments, the microscopic camera is configured to capture bright-field microscopic images and/or fluorescent images of the blood sample. In embodiments including the imager 142, the processor 180 receives signals from the imager 142 indicative of images of a blood sample. In these embodiments, the blood sample need not flow through a cuvette 115 as described above and depicted in FIGS. 1, 2A, and 2B. Instead, images can be taken of a stationary blood sample, and the images can be analyzed by the processor 180.

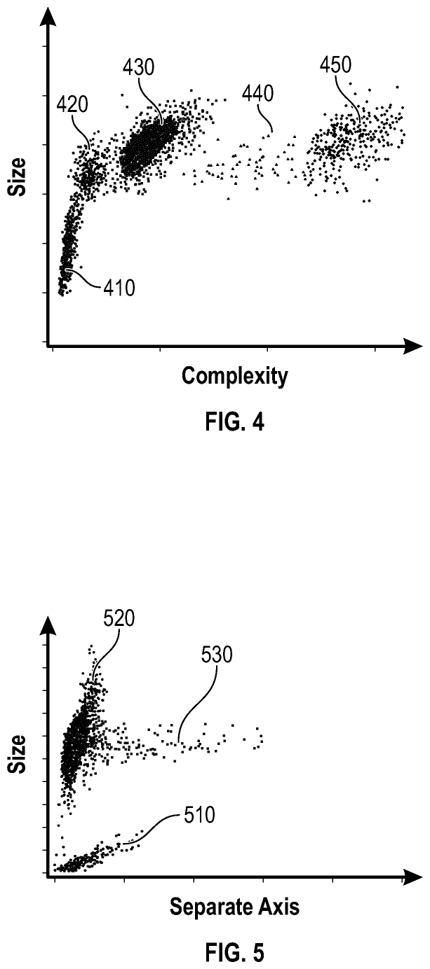

Referring again to FIG. 1, and as will be described in more detail below, the analyzer 150 may generate a two-dimensional (2D) dot plot that presents the constituents of a sample as dots in a two-dimensional (2D) plot, and the analyzer 150 may provide automated analysis, instructions, or other information based on the spatial distribution of one or more groups of dots in the 2D dot plot. The analyzer 150 may provide the automated analysis (and optionally the 2D dot plot) to the output device 160 to be presented to a user. Examples of 2D dot plots are shown in FIGS. 4 and 5, which are described below. The QC material 170 of FIG. 1 will be described in more detail later in connection with the 2D dot plots. For now, it is sufficient to note that the QC material 170 may be used by the hematology system 100 to characterize the system 100's behavior, and the analyzer 150 can use the characterization to normalize the 2D dot plots it generates. FIGS. 1-3 and their corresponding descriptions are provided merely as examples and are not intended to limit the scope of the present disclosure.

In accordance with aspects of the present disclosure, and with reference to FIGS. 1-5, the illustrated 2D dot plots have one axis that represents values of complexity of the constituents in a sample and another axis that represents values of size of the constituents in a sample.

In embodiments, the complexity axis of the 2D dot plot corresponds to sensed data of one or more sensors in the hematology system 100 or corresponds to a complexity metric that indicates the complexity of the constituent cells in a hematology sample (e.g., cell shape, degree of development of the nucleus, granules, RNA/DNA, of the constituent cells, etc.). In some embodiments, the complexity is a quantity that is derived from sensed data. For example, the complexity may be a quantity that is computed as a function of sensed SS data, sensed FSL data, sensed FSH data, sensed EXT data, sensed TOF data, sensed fluorescence data, image data from the imager 142, and/or other sensed data. Persons skilled in the art will understand complexity and how to compute complexity.

In embodiments, size is represented by one axis of the 2D dot plot and is a quantity that is derived from the sensed data and/or metrics of the hematology system. Persons skilled in the art will understand how to indicate size of constituents using sensed data and/or metrics. For example, the size of cells 120 may be determined based on FSL and/or EXT data. Without being bound by theory, EXT and FSL sensor signals both have strong sensitivity to size of constituents in a hematology sample, and either signal can be used to indicate size of such constituents. In embodiments, the size of particular constituents (e.g., red blood cell, platelet, etc.) may be indicated using either the E×T sensor signal or the FSL sensor signal. In embodiments, the size of particular constituents may be indicated by considering both the EXT and the FSL sensor signals. The EXT and FSL sensor signals are merely examples, and other sensed data and/or metrics may be used to indicate size. In embodiments in which the hematology system 100 includes an imager 142, size is a quantity that is derived from image data. Persons skilled in the art will understand how to derive size of a constituent from image data. For example, the geometric extents of a cell may be identified and size may be determine based on known magnification and pixel resolution, impact of reagents on spherical nature of cells, and/or other factors.

With continuing reference to FIG. 4, the illustrated 2D dot plot presents white blood cells processed by the hematology system 100. As persons skilled in the art will understand, white blood cells can include various types of cells, including lymphocytes, monocytes, neutrophils, basophils, and eosinophils, and these types of cells may have different sizes and complexities. In this configuration, larger cells will generally appear higher on the size axis, and cells with more complexity (e.g., more irregular shapes, further developed nucleus, granules, RNA/DNA, etc.) will generally appear farther right on the complexity axis. When healthy white blood cells are plotted in a 2D dot plot, the different types of white blood cells may present themselves relative to each other as shown in FIG. 4. In the example depicted in FIG. 4, the first group of dots 410 are lymphocytes, the second group of dots 420 are monocytes, the third group of dots 430 are neutrophils, the fourth group of dots 440 are basophils, and the fifth group of dots 450 are eosinophils. An example of a system that can generate the type of 2D dot plot shown in FIG. 4 is the IDEXX ProCyte One hematology analyzer. The 2D dot plot of FIG. 4 and the particular size and complexity described above are merely examples, and other 2D plots with different values indicative of healthy cells are contemplated. As one example and without being bound by theory, different species have different size and complexity values for healthy blood cells. In particular, the 2D plot shown in FIG. 4 is of a healthy canine subject, while the lymphocytes, monocytes, neutrophils, basophils, and eosinophils of a healthy feline subject may have different sizes and complexities along the size axis and the complexity axis.

Referring now to FIG. 5, the illustrated 2D dot plot presents red blood cells processed by a hematology system. As persons skilled in the art will understand, platelets and reticulocytes appear with red blood cells. Platelets are a component of blood that play a critical role in normal and abnormal hemostasis, and reticulocytes are immature red blood cells. Platelets, reticulocytes, and red blood cells may have different size, complexity, and/or fluorescence. In FIG. 5, size is represented by one axis of the 2D dot plot, and the separate axis may be indicative of complexity or fluorescence. When healthy red blood cells, platelets, and reticulocytes are plotted in such a 2D dot plot, the different cells may present themselves relative to each other as shown in FIG. 5. The first group of dots 510 are platelets, the second group of dots 520 are red blood cells, and the third group of dots 530 are reticulocytes. In the illustrated embodiment, the group of dots 520 corresponding to red blood cells does not include red blood cell fragments, such as fragments resulting from lysed red blood cells. An example of a system that can generate the type of 2D dot plot shown in FIG. 5 is the IDEXX ProCyte Dx hematology analyzer. The 2D dot plot of FIG. 5 and the size, complexity, and florescence described above are merely examples. For example, and without being bound by theory, different species may have different sizes, complexity, and fluorescence responses for healthy blood samples. In particular, the 2D dot plot shown in FIG. 5 is of a healthy canine, while a blood sample from a healthy feline would have a different size, complexity, and fluorescence values. Further, red blood cells, platelets, and reticulocytes can be plotted in a 2D dot plot having a size axis and another metric or sensed data for the separate axis, as opposed to fluorescence or complexity for the separate axis. The metric or sensed data represented by the separate axis does not need to be (but may be) orthogonal to the size represented by the size axis. Such and other embodiments are contemplated to be within the scope of the present disclosure.

Referring again to FIGS. 1-3, the hematology system 100 may use sensed data for SS, FSL, FSH, EXT, TOF, image data, fluorescence data, and/or use other sensed data, to assign a constituent type to each constituent (e.g., each cell) of a sample. For example, with regard to a hematology sample, the constituent types may include lymphocytes, monocytes, neutrophils, basophils, eosinophils, red blood cells, reticulocytes, and platelets, among others. In embodiments, the constituent types may include red blood cell fragments, such as fragments resulting from lysed red blood cells. The assignment of a constituent type may be performed by algorithms. In embodiments, the algorithms may include heuristic rules developed based on observations of experts. In embodiments, the algorithms may apply machine learning techniques by which a human expert identifies what the reference truth is for the constituent type of constituents in a sample, and a machine learning algorithm develops the associated model. A combination of these approaches can be implemented by, for example, applying heuristic rules before the machine learning model to simplify the data for the machine learning model or by applying heuristic rules after the machine learning model to make adjustments in cases where the machine learning model can be inaccurate due to situations that are not well represented in the machine learning training set.

The assignment of a constituent type to a constituent (e.g., a cell) does not mean and is not intended to mean that the assigned type for each detected cell is correct without error. Rather, as mentioned above, the assignment of a constituent type may be performed using heuristic rules, algorithms, and/or machine learning techniques, among other approaches, which have some error rate. A sufficiently low error rate, however, will provide confidence in the assigned constituent types. Examples of systems which assign constituent types to the constituents of a sample are the IDEXX ProCyte Dx hematology analyzer and the IDEXX ProCyte One hematology analyzer.

With reference again to FIGS. 4 and 5, the hematology system 100 generates the 2D dot plots based on the sensed data. The 2D dot plots generated by different systems may be non-identical even when they analyze the same thing (i.e., the same blood sample). This may be caused by, for example, small variations in each hematology system. For example, in embodiments in which the hematology system 100 is a flow cytometer, variations related to optical path differences within the laser module (e.g., due to slight alignment variations or imperfections in optics) and/or slight fluidic variations, among other reasons, can cause variations between hematology systems 100. Similarly, in embodiments in which the hematology system 100 is an image-based system with the imager 142, variations may exist between imagers 142 of different systems. These variations may be characterized and accounted for using quality control procedures.

In accordance with aspects of the present disclosure, and with reference also to FIGS. 1-3, the hematology system 100 may utilize one or more QC materials 170 that have known properties, e.g., known morphology, size, interactivity with light, and/or the like. Accordingly, as the QC materials 170 are interrogated by the hematology system (e.g., via flow cytometry or imaging) the QC materials 170 should present on a 2D plot in a known manner. The hematology system 100 may store reference values of sensed data for the QC material(s) 170, may store a reference 2D dot plot that shows known locations of dots for the QC material(s) 170, or may store other reference information relating to the known properties of the QC material(s) 170. At various intervals, the hematology system 100 may interrogate QC material(s) 170 (e.g., via flowing the QC material(s) through the flow cell 115 or via imaging the QC material(s) with the imager 142). Information generated from this interrogation can be compared to the reference information to determine adjustments that can normalize the sensed data and/or the dots in a 2D dot plot to match the reference information.

In some embodiments, subtle differences related to the sample path in the hematology system 100 may also affect 2D dot plots, and such differences may not be captured by the QC materials 170. Rather, quality control that accounts for such variations may be performed based on the cells present in the sample. Adjustments may be made on a sample-by-sample basis to account for variables for that specific sample and to normalize the 2D dot plot. An example of such quality control is described in U.S. Patent Application Publication No. US20150025808A1, which is hereby incorporated by reference herein in its entirety.

In embodiments, the adjustments described above may be computed by the analyzer 150, and the analyzer 150 may apply the adjustments to sensed data and/or to the dots in 2D dot plot for a patient sample to normalize the 2D dot plot. Normalizing the 2D dot plot to account for differences between hematology systems allows the various analyses to not be influenced by instrument-specific factors. The normalization measures described above are merely examples. Other normalization measures are contemplated to be within the scope of the present disclosure, including various measures described in U.S. Pat. No. 11,441,997, which is hereby incorporated by reference herein in its entirety.

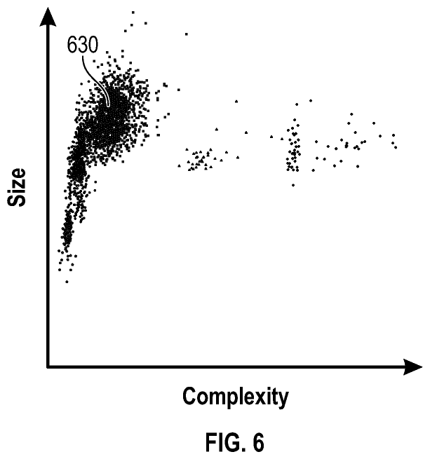

The following will describe analyses relating to the spatial distribution of groups of dots in a 2D dot plot to determine the presence of various conditions in a blood sample, such as left shift or the presence of small pathologic red blood cells. As used herein, the term “spatial distribution” refers to and includes any characterization of the space occupied by a group of dots in a 2D dot plot, including characterizations such as shape, orientation, spread, positioning relative to a configurable position, standard deviation along one or both axes, and density, among other characterizations. Various spatial distribution analyses are described below. FIGS. 6-10 relate to analyzing a 2D dot plot to determine the presence of left shift. FIGS. 11-14 relate to analyzing a 2D dot plot to determine the presence of small-pathologic red blood cells.

Without being bound by theory, one indication of inflammation is that white blood cell populations in a blood sample contain a higher proportion of immature cells. For example, white blood cell populations in a blood sample from a subject with inflammation may have a higher proportion of immature neutrophils, which occurs as inflammatory cytokines stimulate bone marrow to produce neutrophils and release mature and immature neutrophils into the blood. Toxic change in neutrophils is another finding that is associated with inflammation.

Indications of inflammation as described above can generally be identified by manual human analysis of blood films under a microscope. For example, a skilled laboratory technician can identify and quantify immature neutrophils and toxic neutrophils. Immature forms of neutrophils may be manually identified by their maturation stage using blood films. The maturation stages from most to least mature are as follows: mature segmented neutrophils, bands, metamyelocytes, myelocytes, promyelocytes and myeloblasts. When inflammation occurs, less mature forms can be present in the blood films Inflammation also produces toxic change in neutrophils in the form of morphologic changes in the cytoplasm (e.g., increased basophilia, vacuolation, granulation, Dohle bodies) and can result in the presence of larger neutrophils if nuclear divisions are skipped.

Accurately recognizing immature neutrophils and toxic change on blood films requires significant training, and even then, accurate identification and quantification via a human review of a blood film can still be plagued by individual subjectivity. In addition, immature neutrophils and toxic change can sometimes be evident in only a small subset of neutrophils present in the sample and may, therefore, be easy to miss in the blood film if a cursory evaluation is performed.

Moreover, in many circumstances, blood films are not typically prepared as part of a blood analysis. Instead, many practitioners rely on results from a hematology system 100 to provide an initial analysis before proceeding to prepare a blood film. However, many practitioners are not familiar with 2D dot plots and may have difficulty accurately identifying conditions indicative of inflammation. Consequently, they may fail to prepare blood films for analysis. Moreover, morphologic characteristics seen with immature neutrophils and toxic change occur as a continuum, and artifactual changes that mimic immature neutrophils and toxic change can occur in aged samples. As such, failure to timely prepare and analyze blood films can result in an inability to correctly identify conditions indicative of inflammation.

The present disclosure provides automated analyses of 2D dot plots to identify conditions indicative of inflammation. By automatically identifying conditions indicative of inflammation, systems and methods according to the present disclosure can assist in identifying circumstances in which further analysis (e.g., preparation of blood slides) should be performed. In some instances, systems and methods according to the present disclosure can provide additional benefits that supplement manual blood film reviews. For example, an analyzer according to the present disclosure may evaluate thousands of cells or more without human intervention. In contrast, generally about one-hundred white blood cells are reviewed for a manual blood film evaluation by a laboratory technician. The following will describe analyzing spatial distribution of neutrophil dots to identify conditions associated with inflammation, without manual examination or human intervention. However, it is intended and contemplated that aspects of the present disclosure for identifying conditions associated with inflammation may be applied to any type of white blood cell.

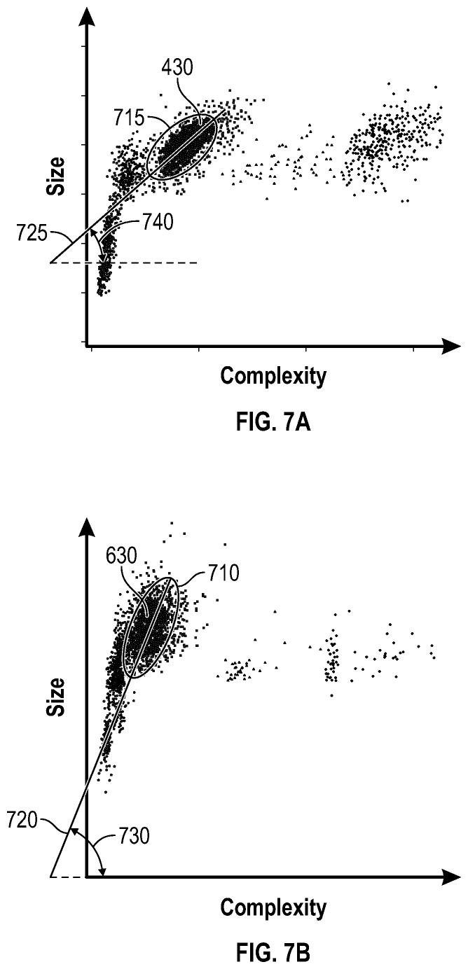

Referring to FIG. 6, in blood samples indicating inflammation, the neutrophils 630 tend to be larger and less complex than normal neutrophils, so in the 2D dot plot, the dots for the neutrophils 630 tend to shift up and to the left along the depicted axes, which is referred to herein as “left shift.” This can be seen in comparing, for example, the spatial distribution of neutrophil dots 430 of FIG. 4 (no inflammation) with the spatial distribution of neutrophil dots 630 of FIG. 6 (inflammation). FIGS. 7A-10 relate to various ways of analyzing the spatial distribution of neutrophil dots to identify left shift. One or more of the analyses described below may be used. Although they are described in relation to neutrophil dots, it is intended that the analyses may be applied to other white blood cells in a similar way.

FIGS. 7A and 7B depict analysis of the spatial distribution of neutrophil dots by determining orientation and dimensions of a geometric shape that surrounds dots of the neutrophil dots 630 in the 2D dot plot. The 2D dot plot of FIG. 7A is a 2D dot plot corresponding to no inflammation. The 2D dot plot of FIG. 7B is a 2D dot plot corresponding to inflammation. In FIGS. 7A and 7B, the geometric shape 710, 715 is an ellipse that defines a major axis 720, 725. The orientation and dimensions of the geometric shape 710, 725 may be determined in various ways. For example, in embodiments in which the geometric shape is an ellipse, the ellipse can be determined via best fit. As one example, starting with the centroid of the neutrophil dots 630, multiple ellipses of different sizes and orientations may be evaluated to determine how much of the neutrophil dots 630 and how much of the other dots are encompassed by the ellipses. The ellipse which best balances a high percentage of neutrophil dots with a low percentage of other dots may be selected as the geometric shape 710. Similarly, in some embodiments, a least squares approach may be utilized to determine the geometric shape. In some embodiments, the geometric shape 710 can be determined by forming best fit ellipse of a predetermined confidence (e.g., 85% confidence, 90% confidence, 95% confidence, 97% confidence, etc.). Other ways of determining the orientation and dimensions of the geometric shape 710 are contemplated to be within the scope of the present disclosure.

In accordance with aspects of the present disclosure, a determination of left shift may be based on the angle 730 formed between a line containing the major axis 720 of the geometric shape 710 and the complexity axis or line parallel to the complexity axis. For explanatory purposes, comparing FIG. 7A (no inflammation) and FIG. 7B (inflammation), the angle 730 is larger than the corresponding angle 740 formed based on the major axis 725 of the geometrical shape 715 around the neutrophil dots 430 corresponding to no inflammation. In embodiments, presence of left shift can be determined in circumstances in which the angle 730 is greater than a configurable threshold angle. For example, the configurable threshold angle, in some embodiments, is determined empirically (e.g., by a human) and may correspond to user input to the hematology system 100 (FIG. 1). In some embodiments, the configurable threshold angle is determined analytically (e.g., by a computer) and may be stored in the memory 184 (FIG. 3) of the hematology system 100. In some embodiments, the configurable threshold angle is a suitable angle for determining left shift and can be an angle less than about 90°. In embodiments, the configurable threshold angle may be about 30°, about 45°, about 60°, or any other suitable threshold angle for indicating left shift.

The approach described in connection with FIGS. 7A and 7B is merely an example. In embodiments, another axis of the geometric shape and/or another axis of the 2D dot plot may be used to form an angle different from angle 730. In embodiments, such an angle may be an angle between a minor axis (not shown) of the geometric shape 710 and the complexity axis. In embodiments, such an angle may be an angle between a minor axis of the geometric shape 710 and the size axis. In some embodiments, left shift may be determined to be present if such an angle (e.g., angle between minor axis and complexity axis) is greater than a configurable threshold angle. In embodiments, left shift may be determined to be present if such an angle (e.g., angle between minor axis and size axis) is less than a configurable threshold angle. Such and other angles may be used to determine presence of left shift, and they are contemplated to be within the scope of the present disclosure.

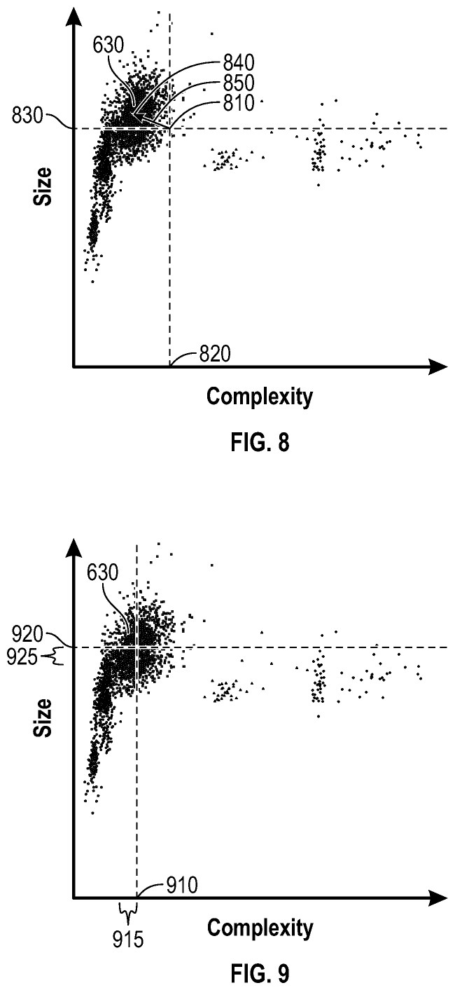

FIG. 8 depicts analysis of the spatial distribution of neutrophil dots by determining a centroid of the neutrophil dots relative to a configurable location in the 2D dot plot where a centroid of healthy neutrophil dots would be located in the 2D dot plot. As mentioned above, when inflammation occurs, the neutrophils tend to be larger and less complex than normal neutrophils, so in the 2D dot plot, the dots for the neutrophils tend to shift up and to the left. This results in a different centroid in the 2D dot plot from the centroid of healthy neutrophil dots. The centroid 810 of healthy neutrophil dots can be, for example, the centroid of the neutrophil dots 430 of FIG. 4. The centroid 810 has a value 820 on the complexity axis (e.g., a healthy complexity value 820) and a value 830 on the size axis (e.g., a healthy size value 830), which can be stored as the configurable complexity and size values. While the healthy complexity value 820 and the healthy size value 830 are depicted as being singular values, it should be understood that this is merely an example, and the healthy complexity value 820 and the healthy size value 830 can include ranges of healthy size and complexity values.

In accordance with aspects of the present disclosure, determination of the presence of left shift is based on the direction (and optionally magnitude) of a vector 850 from the configurable location 810 to a centroid 840 of the neutrophil dots 630. Persons skilled in the art will understand how to determine direction and magnitude of the vector 850. In some embodiments, the analyzer 150 (FIG. 1) determines the presence of left shift based at least in part in response to determining that the direction of the vector 850 is towards the lesser value side of the healthy complexity value 820. For example, in some embodiments, the analyzer 150 determines the presence of left shift in response to determining that the direction of the vector 850 is toward the lesser-value side of the healthy complexity value 820 and based on the vector 850 having a magnitude greater than a configurable threshold. In some circumstances, the analyzer 150 may determine the presence of left shift in response to determining that the direction of the vector 850 is to a greater-value side of the healthy size value 830. For example, in some embodiments, the analyzer 150 determines the presence of left shift based at least in part in response to determining that the direction of the vector 850 is toward the greater-value side of the healthy size value 830 and based on the vector 850 having a magnitude greater than a configurable threshold. In some embodiments, the analyzer 150 determines the presence of left shift based at least in part on the direction of the vector 850 being toward the lesser-value side of the healthy complexity value 820 and being toward the greater-value side of the healthy size value 830 and based on the vector 850 having a magnitude greater than a configurable threshold in the complexity and the size directions. The configurable magnitude threshold may be determined in any suitable manner, for example empirically (e.g., by a human) and/or analytically (e.g., by a computer).

The approach described in connection with FIG. 8 is merely an example. In embodiments, the approach of FIG. 8 may be used in conjunction with or as an alternative to the approach of FIGS. 7A/7B. For example, in embodiments, hematology systems 100 according to the present disclosure may determine the presence of left shift utilizing one or both of the approaches outlined above and depicted in FIGS. 7A/7B and 8.

FIG. 9 relates to analyzing the spatial distribution of neutrophil dots by determining standard deviation (and optionally mean) of the neutrophil dots along the size and complexity axes. FIG. 9 shows an example of the mean 910 and standard deviation 915 of the neutrophil dots 630 along the complexity axis and the mean 920 and standard deviation 925 of the neutrophil dots 630 along the size axis. Persons skilled in the art will understand how to determine mean and standard deviation.

In accordance with aspects of the present disclosure, presence of left shift can be determined based on one or both of the standard deviations 915, 925. In embodiments, left shift is determined to be present if the standard deviation 915 along the complexity axis is less than a complexity standard deviation threshold. In embodiments, left shift is determined to be present if the standard deviation 925 along the size axis is greater than a size standard deviation threshold. In embodiments, left shift is determined to be present if the standard deviation 915 with respect to the complexity axis is less than the complexity-axis standard deviation threshold and if the standard deviation 925 with respect to the size axis is greater than a size-axis standard deviation threshold. The complexity-axis standard deviation threshold and the size-axis standard deviation threshold may be determined empirically (e.g., by a human) and/or analytically (e.g., by a computer) and may be stored as a configurable complexity-axis standard deviation threshold and as a configurable size-axis standard deviation threshold, respectively.

The approach described in connection with FIG. 9 is merely an example. In embodiments, the approach of FIG. 9 may be used in conjunction with or as an alternative to the approaches of FIGS. 7A/7B and/or FIG. 8. For example, in embodiments, left shift may be determined to be present if any approach indicates that left shift is present. In embodiments, left shift may be determined to be present only if two or more approaches or if all of the approaches indicate that left shift is present.

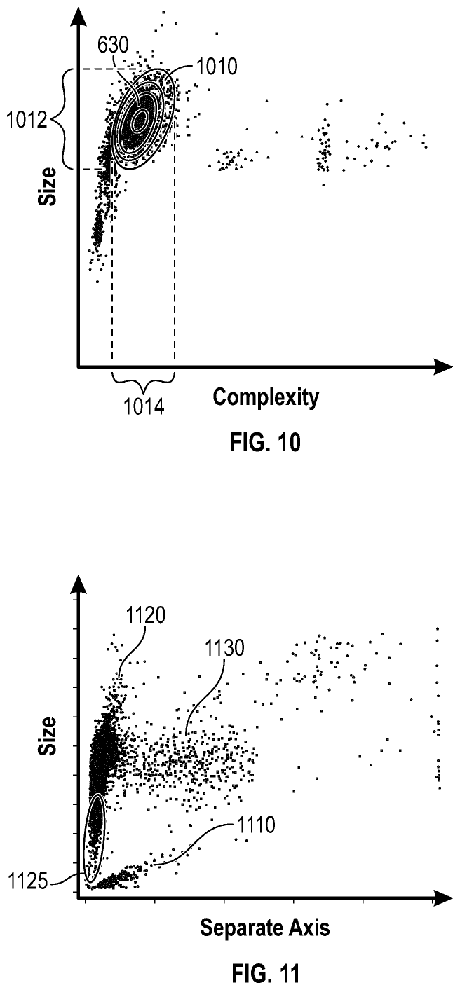

FIG. 10 relates to analyzing the spatial distribution of the neutrophil dots by determining spatial density bands of the neutrophil dots in the 2D dot plot. FIG. 10 shows multiple density bands of neutrophil dots 630, including a low-density band 1010. As used herein, the term “density” refers to the number of dots in a unit area of a 2D dot plot. Density of the neutrophil dots 630 may be determined in various ways, including, for example, by overlaying a grid (not shown) onto the 2D dot plot and determining the number of neutrophil dots in each box of the grid. The number of spatial density bands and the density range of each band may be configured in various ways. In embodiments, the lowest density band may be designated as the low-density band 1010 for determining left shift. In embodiments, a density band other than the lowest density band may be designated as the low-density band 1010 for determining left shift.

In accordance with aspects of the present disclosure, left shift may be determined to be present in circumstances in which the designated low density band 1010 defines a height 1012 (evaluated along the size axis) that is greater than a configurable height threshold and/or a width 1014 (evaluated along the complexity axis) that is less than a configurable width threshold. Similar to the approach described above and depicted in FIG. 9, the height 1012 of the designated low density band 1010 (evaluated along the size axis) may correlate to a standard deviation of the size of constituent cells, and the width 1014 of the designated low density band 1010 may correlate to a standard deviation of the complexity of constituent cells.

The approach described in connection with FIG. 10 is merely an example. In embodiments, other analyses of density bands are contemplated, such as comparing density bands to a band size threshold. In embodiments, the approach of FIG. 10 may be used in conjunction with or as an alternative to the approaches of FIGS. 7A-9. For example, in embodiments, left shift may be determined to be present if any approach indicates there is left shift. In embodiments, left shift may be determined to be present only if two or more approaches or if all of the approaches indicate that left shift is present.

In accordance with aspects of the present disclosure, another approach to determining presence of left shift can apply machine learning techniques to identify the presence of immature and or toxic neutrophils. For this approach, an expert at evaluating 2D dot plots and blood films for manual confirmation of inflammation could classify/designate a population of dot plots from various samples. The 2D dot plots could be designated as present or absence of immature and or toxic neutrophils, or they could be classified further into a semi-quantitative bucketing system, such as absent, mild, moderate, or significant for immature and or toxic neutrophil presence. In embodiments, the level of immaturity and or toxic change could be quantified as the number of cells (e.g., bands, other neutrophil precursor cells) and degree of toxicity noted in the sample per one-hundred white blood cells. The training performs machine learning calculations that will build a model to predict the presence of immature and or toxic neutrophils to the granularity that is presented in the reference data. The machine learning approach continues this supervised training approach until a pre-defined error performance is achieved and the algorithm can be considered trained. Verification with samples that were not used as part of the training set can confirm the efficacy of the algorithm with standard acceptance criteria including statistics, such as sensitivity and specificity, as well as confusion matrices if the reference is semiquantitative, or even regression statistics such as slope and correlation coefficient if the data is quantitative. Other metrics such as repeatability can also be demonstrated before the algorithm is accepted. The machine learning approach may be used in conjunction with or as an alternative to the approaches of FIGS. 7A-10. The description above is merely an example, and other machine learning approaches may be applied. Persons skilled in the art will understand how to implement such approaches.

Accordingly, described above are approaches for analyzing spatial distributions of white blood cell dots in 2D dot plots to determine presence of left shift. The approaches described above may be applied to different hematology systems that use data that can be presented in different 2D dot plots. In FIGS. 4 and 6-10, the horizontal axis is depicted as showing complexity, but it should be understood that the horizontal axis may correspond to florescence or another suitable metric or sensed data. The approaches described above may also be applied to hematology samples of different species, such as cats, dogs, and other species. The various thresholds described above for determining presence of left shift may have different threshold values for different species, but otherwise, the approaches are applicable for determining presence of left shift in any patient.

The following paragraphs will describe, in connection with FIGS. 11-15, approaches for analyzing spatial distributions of red blood cells in 2D dots plots to determine presence of small-pathologic red blood cells (SP-RBC). The 2D dot plots of FIGS. 11-15 include a size axis and a separate axis that may correspond to complexity or fluorescence, among other data or metrics. However, the disclosed approaches are applicable to any 2D dot plot. The approaches described below are also applicable to determining presence of SP-RBC in different species, but various thresholds in the different approaches may have different threshold values for different species and for different 2D dot plots.

Small-pathologic red blood cells are typically identified after examining a blood film. Due to the various mechanisms that produce SP-RBC, these cells have different morphologies that can guide identification of the underlying pathologic process. Identification of distinct red blood cell morphology changes can indicate underlying nonspecific disease or lead directly to identification of the specific primary pathologic process.

As an example, immune-mediated hemolytic anemia (IMHA) is a condition where anemia results from immune-mediated destruction of red blood cells. During this process, in the majority of cases, antibody coats the red cells, which signals macrophages to remove a portion of the red cell membrane. As macrophages extract pieces of the membrane, spherocytes (smaller appearing red cells with decreased central pallor) are produced. Initially, these spherocytes are similar in size to normal red blood cells since primarily cell membrane is lost and overall cell volume remains normal; however, as these cells interact with the macrophages, greater and greater amounts of cytoplasm is lost and the overall red blood cell size decreases. Spherocytes are a key diagnostic feature of IMHA and have been reported to occur in up to 90% of dogs with IMHA. Identifying many spherocytes can lead the clinician to make critical therapeutic decisions for treating the anemic patient.

As another example, oxidative injury to red blood cells results from exposure to some drugs (e.g., acetaminophen), oxidative agents (onions, zinc), and in association with certain disease processes (e.g., neoplasia, diabetes). Oxidative injury can denature hemoglobin which produces Heinz bodies, or damage red cell membranes, generating eccentrocytes, blister cells and keratocytes. All mechanisms result in smaller than normal erythrocytes. When oxidative injury is marked, it can result in secondary hemolytic anemia. If the anemia is primarily the result of oxidative damage, identification and removal of the inciting cause is crucial for treatment.

With regard to metabolic and membrane disorders, there are nonspecific red blood cell changes that can occur secondary to alternations or injury of the red cell membranes. Although the changes are nonspecific, they can indicate underlying disease that could otherwise be undetected. Certain morphologies can suggest a selected list of more common differentials that can aid the clinician's diagnostic choices. Blister cells/keratocytes occur after alterations or injury to the red blood cell membrane and can be associated with different underlying causes (e.g., iron deficiency, oxidative injury, liver disease, microangiopathic disease). Acanthocytes are thought to be produced by alterations in the lipid composition of the red cell membranes or mechanical fragmentation. They are an important indicator of underlying disease and in canines have been associated with a number of processes (e.g., cancer, liver disease, iron deficiency and disseminated intravascular coagulation (DIC)). Lastly, poikilocytosis in feline patients can signal metabolic disease (e.g., liver disease, renal disease, hyperthyroidism) and should prompt further diagnostics when present in significant numbers.

With regard to mechanical injury, schistocytes are red cell fragments and they reflect mechanical injury to red cells. They often form when fibrin strands are present within the microvasculature or when vascular disease results in an abnormal endothelial lining or turbulent blood flow. Some examples of conditions in which schistocytes occur are DIC, vasculitis and hemangiosarcoma. As schistocytes result from fragmentation, they can also occur when other pathologic processes result in the production of red cells with increased mechanical fragility (e.g., secondary to iron deficiency, alternations in red cell membranes).

Iron deficiency can occur because of an iron-deficient diet. However, in canine and feline patients, most cases of iron deficiency result from chronic external blood loss (e.g., gastrointestinal, urinary hemorrhage, parasites). Decreased iron availability will affect erythroid production resulting in smaller cells (microcytes) and cells with reduced hemoglobin concentration (hypochromic cells). Microcytic and hypochromic erythrocytes are key indicators for iron deficiency and cue clinicians to search for underlying causes of blood loss. Capturing concurrent red cell morphology changes significantly aids specificity. After determining a patient has iron deficiency anemia, appropriately chosen diagnostics can expose the primary disease that is resulting in chronic external blood loss. (e.g., neoplasia, ulcers, parasitism)

Because small-pathologic red blood cells are typically identified after examining a blood film, SP-RBC has not been diagnosed by point-of-care hematology analyzers. The present disclosure provides automated analyses of 2D dot plots to determine presence of SP-RBC and provides benefits over manual blood film reviews and existing hematology analyzers. As described above, pathologic changes in red blood cells can result from a variety of causes, and many of the mechanisms ultimately result in the generation of smaller red blood cells that have decreased cell volume. Different species, such as cats and dogs, will have different size red blood cells, but they generally show a clear distribution of red blood cells exhibiting SP-RBC.

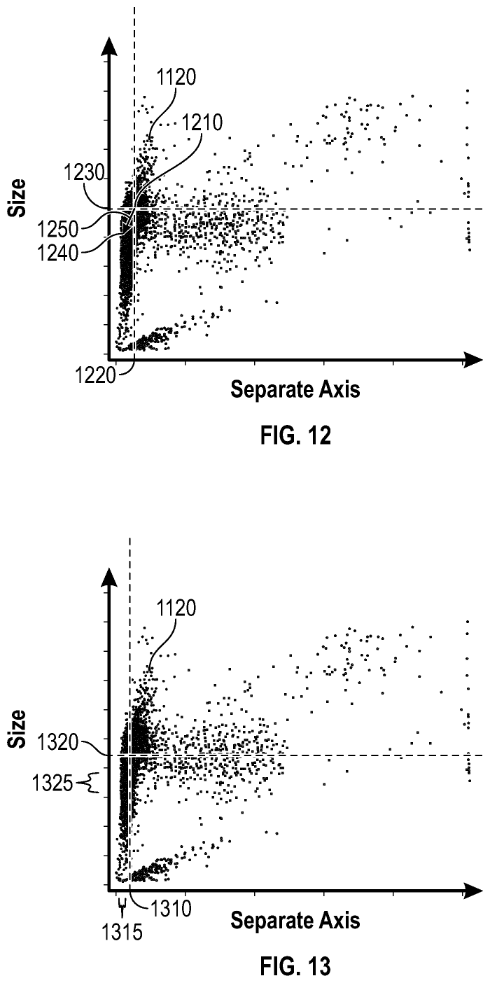

For example, FIG. 11 shows a 2D dot plot of platelets 1110, red blood cells 1120, reticulocytes 1130, in a patient that has SP-RBC. When SP-RBC are present, they form a specific population 1125 that falls below the natural, mature RBC population and stretches towards the platelet population 1110. Compare, for example, the red blood cell dots 520 of FIG. 5 (no SP-RBC), which are not stretched towards the platelet population 510. Further, this population of red blood cell dots 1125 that stretches towards the platelet population 1110 is distinct from red blood cell fragments that may have lysed during the sample preparing process. FIGS. 12-14 relate to various ways of analyzing the spatial distribution of red blood cell dots to determine presence of SP-RBC. One or more of the analyses described below may be used to determine presence of SP-RBC.

FIG. 12 relates to analyzing the spatial distribution of red blood cell dots by determining a centroid of the red blood cell dots 1120 relative to a configurable location 1210 in the 2D dot plot where a centroid of dots corresponding to healthy populations of red blood cells would be located in the 2D dot plot. The configurable location/centroid 1210 of healthy red blood cell dots can be, for example, the centroid of the red blood cell dots 520 of FIG. 5. The centroid 1210 has a value 1220 on the separate axis (e.g., a separate-axis value 1220) and a value 1230 on the size axis (e.g., a size-axis value 1230), which can be stored as the configurable location 1210 in the 2D dot plot.

In accordance with aspects of the present disclosure, presence of SP-RBC may be determined based on the direction and magnitude of a vector 1250 from the configurable location 1210 to the centroid 1240 of the red blood cell dots 1120. Persons skilled in the art will understand how to determine direction and magnitude of the vector 1250. In embodiments, SP-RBC may be determined to be present if the direction of the vector 1250 is towards the lesser-value side of the size-axis value 1230 and if the magnitude of the vector 1250 is greater than a configurable threshold. The magnitude threshold may be determined empirically (e.g., by a human) and/or analytically (e.g., by a computer) and may be stored as a configurable magnitude threshold.

The approach described in connection with FIG. 12 is merely an example, and variations are contemplated to be within the scope of the present disclosure. For example, in embodiments, determining presence of SP-RBC may also consider the direction of the vector 1250 with respect to the separate-axis value 1220; e.g., whether the direction of the vector 1250 is toward a lesser-value side of the separate-axis value 1220. Such and other embodiments are contemplated to be within the scope of the present disclosure.

FIG. 13 relates to analyzing the spatial distribution of red blood cell dots by determining a size-axis distribution of a subset of the dots 1120 below the mean or median of the red blood cell dots 1120 evaluated along the size axis and, optionally, also a separate-axis distribution of a subset of the dots 1120 below the mean or median of the red blood cell dots 1120 evaluated along the separate axis. FIG. 13 shows an example of the separate-axis mean or median 1310 of red blood cell dots 1120 and a size-axis mean or median 1320 of the red blood cell dots 1120. In the example depicted in FIG. 13, a size-axis distribution 1325 of the red blood cell dots 1120 below the size-axis mean or median 1320 along the size axis is depicted. Also, a separate-axis distribution 1315 of the red blood cell dots 1120 below the separate-axis mean or median 1310 along the separate axis is depicted. The distributions 1315, 1325 may correspond to, for example, standard deviation, percentage coefficient of variation, or other metrics. Persons skilled in the art will understand how to determine mean, median, and distribution.

In accordance with aspects of the present disclosure, presence of SP-RBC may be determined based on the size-axis distribution 1325 evaluated for the subset of the dots 1120 below the size-axis mean or median 1320. In embodiments, SP-RBC may be determined to be present if the size-axis distribution 1325 is greater than a configurable size-axis distribution threshold. In embodiments, determining presence of SP-RBC may additionally consider the separate-axis distribution 1315 evaluated for the subset the dots 1120 below the separate-axis mean or median 1310, e.g., in comparison to a configurable separate-axis distribution threshold. The separate-axis distribution threshold and the size-axis distribution threshold may be determined empirically (e.g., by a human) and/or analytically (e.g., by a computer) and may be stored as a configurable separate-axis distribution threshold and as a configurable size-axis distribution threshold, respectively.

The approach described in connection with FIG. 13 is merely an example. In embodiments, the approach of FIG. 13 may be used in conjunction with or as an alternative to the approach of FIG. 12. For example, in embodiments, SP-RBC may be determined to be present if either approach determines that SP-RBC is present. In embodiments, SP-RBC may be determined to be present only if all of the approaches determine that SP-RBC is present.

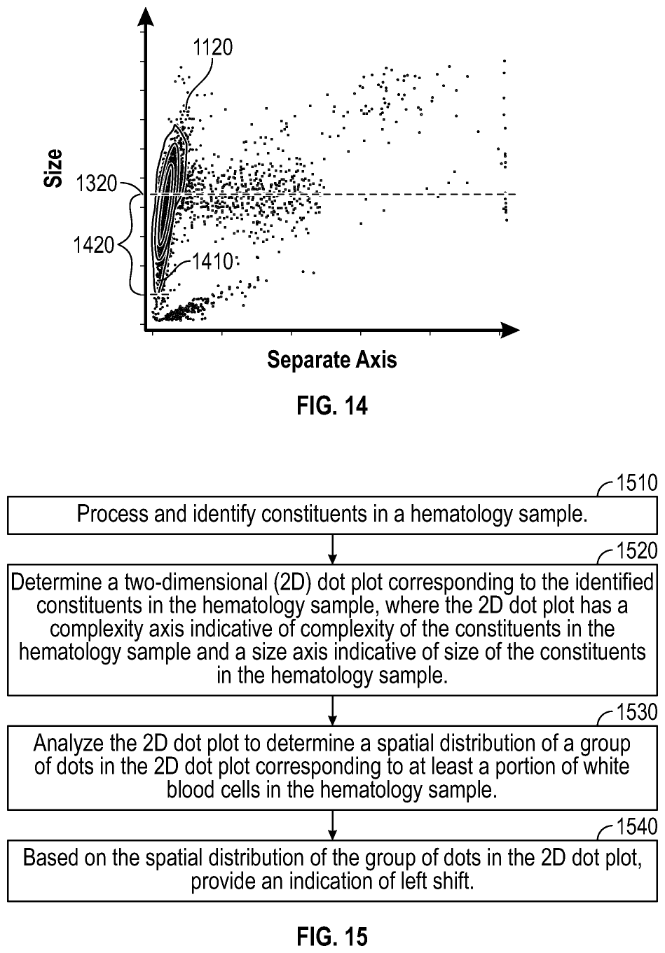

FIG. 14 relates to analyzing the spatial distribution of red blood cell dots by determining spatial density bands of the red blood cell 1120 dots in the 2D dot plot. As mentioned above, the term “density” as used herein refers to the number of dots in a unit area of a 2D dot plot. FIG. 14 shows multiple density bands of red blood cell dots 1120, including a low-density band 1410. Density of the red blood cell dots 1120 may be determined in various ways, including, for example, by overlaying a grid (not shown) onto the 2D dot plot and determining the number of red blood cell dots in each box of the grid. The number of spatial density bands and the density range of each band may be configured in various ways. In embodiments, the lowest density band may be designated as the low-density band 1410 for determining presence of SP-RBC. In embodiments, a density band other than the lowest density band may be designated as the low-density band 1410 for determining presence of SP-RBC.

FIG. 14 depicts a size-axis mean or median 1320 of the red blood cell dots 1120. In accordance with aspects of the present disclosure, SP-RBC may be determined to be present in circumstances in which the designated low-density band 1410 extends below the size-axis mean or median 1320 by a height 1420 that is more than a configurable height threshold. The density range of the designated low-density band 1410 and the configurable height threshold may be determined empirically (e.g., by a human) and/or analytically (e.g., by a computer) and may be stored as a configurable density range and as a configurable height threshold, respectively.

The approach described in connection with FIG. 14 is merely an example. In embodiments, other analyses of density bands are contemplated, such as comparing density bands to a band size threshold or analyzing the reach of a density band along one or both axes. In embodiments, the approach of FIG. 14 may be used in conjunction with or as an alternative to the approaches of FIG. 12 or FIG. 13. For example, in embodiments, SP-RBC may be determined to be present if any approach determines that SP-RBC is present. In embodiments, SP-RBC may be determined to be present if two or more of the approaches or if all of the approaches determine that SP-RBC is present.

In accordance with aspects of the present disclosure, another approach to determining presence of SP-RBC can apply machine learning techniques to identify presence of the population 1125 shown in FIG. 11. For example, manual (visual) analysis of the 2D dot plots provides reference truth for when these cells are present and/or for a semiquantitative measure (mild, moderate, significant, for example) of how much SP-RBC is present, and the reference truth may be used for training a machine learning system. Machine learning approaches can be employed for training a system to automatically identify the presence and relative amount of these cells 1125. The system can identify the portion of cells that represent SP-RBC and divide that into the total RBC count to determine the relative prevalence. This value can then be translated to relevant medical information in a quantitative manner, in semi-quantitative buckets (mild, moderate, significant, for example), or as presence or absence by comparing with a threshold. The end result may provide an indication to the customer regarding the presence of these cells and potential implications of the determination. The machine learning approach may be used in conjunction with or as an alternative to the approaches of FIGS. 12-14. The description above is merely an example, and other machine learning approaches may be applied. Persons skilled in the art will understand how to implement such approaches.

As mentioned above, there are various mechanisms that produce SP-RBC, and these mechanisms result in different morphologies that can guide identification of the underlying pathologic process. In accordance with aspects of the present disclosure, after SP-RBC is determined to be present based on one or more of the analyses described above, a manual blood film analysis may be performed to identify the distinct red blood cell morphology changes and to diagnose the underlying nonspecific disease or the specific primary pathologic process, such as those described above herein.

FIG. 15 is a flow chart of an exemplary operation for providing determining presence of left shift, without human intervention. The operation of FIG. 15 may be performed by one or more processors executing instructions. In embodiments, the operation may be performed in a hematology system, such as in the system 100 of FIG. 1, and may be performed by the analyzer 150 of FIG. 1.

While the approaches described above describe the analysis of data presented in 2D dot plots, it should be understood that systems and methods of the present disclosure do not require the data to be presented in dot plot format to be analyzed. In other words, each of the approaches discussed above may be performed by the analyzer 150 utilizing sensed data, without requiring that the analyzer 150 present a 2D dot plot.

At block 1510, the operation involves, without human intervention, processing and identifying constituents in a hematology sample. The constituents may be processed and identified in the manners described above herein in connection with FIGS. 1-3. At block 1520, the operation involves, without human intervention, determining a two-dimensional (2D) dot plot corresponding to the identified constituents in the hematology sample, where the 2D dot plot has a complexity axis indicative of complexity of constituents in the hematology sample and a size axis indicative of size of the constituents in the hematology sample.

At block 1530, the operation involves, without human intervention, analyzing the 2D dot plot to determine a spatial distribution of a group of dots in the 2D dot plot corresponding to at least a portion of white blood cells in the hematology sample. In embodiments, the group of dots corresponding to white blood cells may be neutrophils. In embodiments, analyzing the spatial distribution of the group of white blood cell dots may include one or more of the analyses described in connection with FIGS. 7A-10 and/or may include machine learning approaches.

At block 1540, the operation involves, without human intervention, providing an indication of left shift based on the spatial distribution of the group of white blood cell dots in the 2D dot plot. In embodiments, the determination may be performed as described in connection with one or more of FIGS. 7A-10 and/or as described in connection with the machine learning approaches. The determination may be provided to an output device (e.g., 160, FIG. 1) and may be provided as a visual message (e.g., displayed on a screen, printed on paper, etc.) and/or as an audio message.

The operation of FIG. 15 is exemplary. The operation of FIG. 15 may be performed in conjunction with other blocks not shown in FIG. 15.

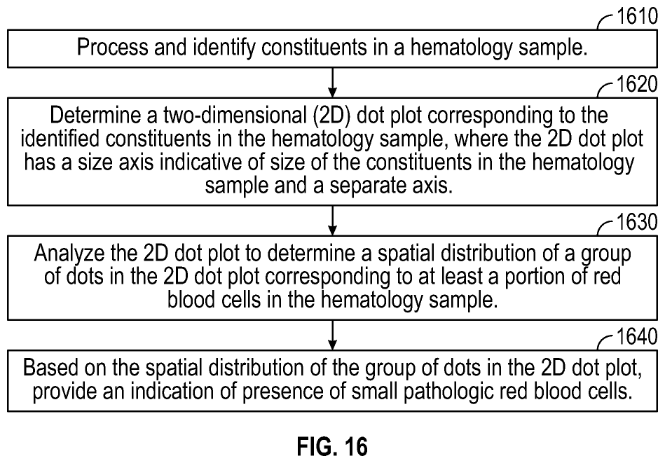

FIG. 16 is a flow chart of an exemplary operation for determining presence of SP-RBC, without human intervention. The operation of FIG. 16 may be performed by one or more processors executing instructions. In embodiments, the operation may be performed in a hematology system, such as in the system 100 of FIG. 1, and may be performed by the analyzer 150 of FIG. 1.

At block 1610, the operation involves, without human intervention, processing and identifying constituents in a hematology sample. The constituents may be processed and identified in the manners described above herein in connection with FIGS. 1-3. At block 1620, the operation involves, without human intervention, determining a two-dimensional (2D) dot plot corresponding to the identified constituents in the hematology sample, where the 2D dot plot has a separate axis and has a size axis indicative of size of the constituents in the hematology sample. In embodiments, the separate axis may correspond to fluorescence or complexity, which were described above.

At block 1630, the operation involves, without human intervention, analyzing the 2D dot plot to determine a spatial distribution of a group of dots in the 2D dot plot corresponding to at least a portion of red blood cells in the hematology sample. In embodiments, analyzing the spatial distribution of the group of red blood cell dots may include one or more of the analyses described in connection with FIGS. 12-14 and/or may include machine learning approaches.

At block 1640, the operation involves, without human intervention, determining presence of small pathologic red blood cells in the hematology sample based on the spatial distribution of the red blood cell dots in the 2D dot plot. In embodiments, the determination may be performed as described in connection with one or more of FIGS. 12-14 and/or as described in connection with the machine learning approaches. The determination may be provided to an output device (e.g., 160, FIG. 1) and may be provided as a visual message (e.g., displayed on a screen, printed on paper, etc.) and/or as an audio message.

The operation of FIG. 16 is exemplary. The operation of FIG. 16 may be performed in conjunction with other blocks not shown in FIG. 16.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The systems, devices, and/or servers described herein may utilize one or more processors to receive various information and transform the received information to generate an output. The processors may include any type of computing device, computational circuit, or any type of controller or processing circuit capable of executing a series of instructions that are stored in a memory. The processor may include multiple processors and/or multicore central processing units (CPUs) and may include any type of device, such as a microprocessor, graphics processing unit (GPU), digital signal processor, microcontroller, programmable logic device (PLD), field programmable gate array (FPGA), or the like. The processor may also include a memory to store data and/or instructions that, when executed by the one or more processors, causes the one or more processors (and/or the systems, devices, and/or servers they operate in) to perform one or more methods, operations, and/or algorithms.

Any of the herein described methods, operations, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

1. A system for identifying conditions in hematology samples, the system comprising:
at least one processor; and
at least one memory storing instructions which, when executed by the at least one processor, cause the system to, without human intervention:
process and identify constituents in a hematology sample;
determine a two-dimensional (2D) dot plot corresponding to the identified constituents in the hematology sample, the 2D dot plot having a complexity axis indicative of complexity of the constituents in the hematology sample and a size axis indicative of size of the constituents in the hematology sample;
analyze the 2D dot plot to determine a spatial distribution of a group of dots in the 2D dot plot corresponding to at least a portion of white blood cells in the hematology sample; and
based on the spatial distribution of the group of dots in the 2D dot plot, provide an indication of left shift.

2. The system of claim 1, wherein in analyzing the 2D dot plot, the instructions, when executed by the at least one processor, cause the system to, without human intervention, perform at least one analysis among a plurality of analyses comprising:
determining orientation and dimensions of a geometric shape that surrounds the group of dots in the 2D dot plot,
determining a centroid of the group of dots relative to a configurable location in the 2D dot plot where a centroid of dots corresponding to healthy populations of corresponding white blood cells would be located in the 2D dot plot,
determining mean and standard deviation of the group of dots with respect to one of: the complexity axis or the size axis, and
determining spatial density bands of the group of dots in the 2D dot plot.

3. The system of claim 2, wherein in analyzing the 2D dot plot, the instructions, when executed by the at least one processor, cause the system to, without human intervention, perform each analysis in the plurality of analyses.

4. The system of claim 2, wherein in providing the indication, the instructions, when executed by the at least one processor, cause the system to, without human intervention:
compare an angle, formed by a major axis of the geometric shape and the complexity axis of the 2D dot plot, to a configurable angle threshold; and
determine presence of left shift based on the angle being greater than the configurable angle threshold.

5. The system of claim 2, wherein the configurable location in the 2D dot plot has a complexity-axis value and a size-axis value,
wherein in providing the indication, the instructions, when executed by the at least one processor, cause the system to, without human intervention:
determine direction and magnitude of a vector from the configurable location to the centroid of the group of dots; and
determine presence of left shift based on:
(1) the direction of the vector being directed towards one or both of:
a lesser-value side of the complexity-axis value, and
a greater-value side of the size-axis value; and
(2) the magnitude of the vector being greater than a configurable magnitude threshold.

6. The system of claim 2, wherein in providing the indication, the instructions, when executed by the at least one processor, cause the system to, without human intervention:
determine presence of left shift based on at least one of: a complexity-axis standard deviation of the group of dots being less than a configurable complexity-axis standard deviation threshold, or a size-axis standard deviation of the group of dots being greater than a configurable size-axis standard deviation threshold.

7. The system of claim 2, wherein in providing the indication, the instructions, when executed by the at least one processor, cause the system to, without human intervention:
determine presence of left shift based on a designated low-density band among the spatial density bands defining at least one of: a height evaluated along the size axis greater than a configurable height threshold or a width evaluated along the complexity axis less than a configurable width threshold.

8. A process-implemented method for identifying conditions in hematology samples, the method comprising, without human intervention:
processing and identifying constituents in a hematology sample;
determining a two-dimensional (2D) dot plot corresponding to the identified constituents in the hematology sample, the 2D dot plot having a complexity axis indicative of complexity of constituents in the hematology sample and a size axis indicative of size of the constituents in the hematology sample;
analyzing the 2D dot plot to determine a spatial distribution of a group of dots in the 2D dot plot corresponding to at least a portion of white blood cells in the hematology sample; and
based on the spatial distribution of the group of dots in the 2D dot plot, providing an indication of left shift.

9. The processor-implemented method of claim 8, wherein analyzing the 2D dot plot comprises, without human intervention, performing at least one analysis among a plurality of analyses comprising:
determining orientation and dimensions of a geometric shape that surrounds the group of dots in the 2D dot plot,
determining a centroid of the group of dots relative to a configurable location in the 2D dot plot where a centroid of dots corresponding to healthy populations of corresponding white blood cells would be located in the 2D dot plot,
determining mean and standard deviation of the group of dots with respect to one of: the complexity axis or the size axis, and
determining spatial density bands of the group of dots in the 2D dot plot.

10. The processor-implemented method of claim 9, wherein analyzing the 2D dot plot comprises, without human intervention, performing each analysis in the plurality of analyses.

11. The processor-implemented method of claim 9, wherein providing the indication comprises, without human intervention:
comparing an angle, formed by a major axis of the geometric shape and the complexity axis of the 2D dot plot, to a configurable angle threshold; and
determine presence of left shift based on the angle being greater than the configurable angle threshold.

12. The processor-implemented method of claim 9, wherein the configurable location in the 2D dot plot has a complexity-axis value and a size-axis value,
wherein providing the indication comprises, without human intervention:
determining direction and magnitude of a vector from the configurable location to the centroid of the group of dots; and
determining presence of left shift based on:
(1) the direction of the vector being directed towards one or both of:
a lesser-value side of the complexity-axis value, and
a greater-value side of the size-axis value; and
(2) the magnitude of the vector being greater than a configurable magnitude threshold.

13. The processor-implemented method of claim 9, wherein providing the indication comprises, without human intervention:
determining presence of left shift based on at least one of: a complexity-axis standard deviation of the group of dots being less than a configurable complexity-axis standard deviation threshold, or a size-axis standard deviation of the group of dots being greater than a configurable size-axis standard deviation threshold.

14. The processor-implemented method of claim 9, wherein providing the indication comprises, without human intervention:
determining presence of left shift based on a designated low-density band among the spatial density bands defining at least one of: a height evaluated along the size axis greater than a configurable height threshold or a width evaluated along the complexity axis less than a configurable width threshold.

15. A non-transitory process-readable medium storing instructions which, when executed by at least one processor of a system, cause the system to, without human intervention:
process and identify constituents in a hematology sample;
determine a two-dimensional (2D) dot plot corresponding to the identified constituents in the hematology sample, the 2D dot plot having a complexity axis indicative of complexity of constituents in the hematology sample and a size axis indicative of size of the constituents in the hematology sample;
analyze the 2D dot plot to determine a spatial distribution of a group of dots in the 2D dot plot corresponding to at least a portion of white blood cells in the hematology sample; and
based on the spatial distribution of the group of dots in the 2D dot plot, provide an indication of left shift.

16. The non-transitory process-readable medium of claim 15, wherein in analyzing the 2D dot plot, the instructions, when executed by the at least one processor, cause the system to, without human intervention, perform at least one analysis among a plurality of analyses comprising:
determining orientation and dimensions of a geometric shape that surrounds the group of dots in the 2D dot plot,
determining a centroid of the group of dots relative to a configurable location in the 2D dot plot where a centroid of dots corresponding to healthy populations of corresponding white blood cells would be located in the 2D dot plot,
determining mean and standard deviation of the group of dots with respect to one of: the complexity axis or the size axis, and
determining spatial density bands of the group of dots in the 2D dot plot.

17. The non-transitory process-readable medium of claim 16, wherein in analyzing the 2D dot plot, the instructions, when executed by the at least one processor, cause the system to, without human intervention, perform each analysis in the plurality of analyses.

18. The non-transitory process-readable medium of claim 16, wherein in providing the indication, the instructions, when executed by the at least one processor, cause the system to, without human intervention:
compare an angle, formed by a major axis of the geometric shape and the complexity axis of the 2D dot plot, to a configurable angle threshold; and
determine presence of left shift based on the angle being greater than the configurable angle threshold.

19. The non-transitory process-readable medium of claim 16, wherein the configurable location in the 2D dot plot has a complexity-axis value and a size-axis value, wherein in providing the indication, the instructions, when executed by the at least one processor, cause the system to, without human intervention:
determine direction and magnitude of a vector from the configurable location to the centroid of the group of dots; and
determine presence of left shift based on:
(1) the direction of the vector being directed towards one or both of:
a lesser-value side of the complexity-axis value, and
a greater-value side of the size-axis value; and
(2) the magnitude of the vector being greater than a configurable magnitude threshold.

20. The non-transitory process-readable medium of claim 16, wherein in providing the indication, the instructions, when executed by the at least one processor, cause the system to, without human intervention:
determine presence of left shift based on at least one of: a complexity-axis standard deviation of the group of dots being less than a configurable complexity-axis standard deviation threshold, or a size-axis standard deviation of the group of dots being greater than a configurable size-axis standard deviation threshold.

21. The non-transitory process-readable medium of claim 16, wherein in providing the indication, the instructions, when executed by the at least one processor, cause the system to, without human intervention:
determine presence of left shift based on a designated low-density band among the spatial density bands defining at least one of: a height evaluated along the size axis greater than a configurable height threshold or a width evaluated along the complexity axis less than a configurable width threshold.

22.-39. (canceled)
